(19) United States
(12) Patent Application Publication
(10) Pub. No.: US 2026/0201341 A1
(43) Pub. Date: Jul. 16, 2026

(54) ORGANOIDS IN EXTRACELLULAR MATRIX DROPLETS

(71) Applicant: President and Fellows of Harvard College, Cambridge, MA (US)

(72) Inventors: David A. Weitz, Cambridge, MA (US); Iris Elke Ruider, Cambridge, MA (US); Jean Carlos Serrano Flores, Cambridge, MA (US); Andreas R. Bausch, Cambridge, MA (US); Perry Ellis, Cambridge, MA (US)

(73) Assignee: President and Fellows of Harvard College, Cambridge, MA (US)

(21) Appl. No.: 19/139,312

(22) PCT Filed: Dec. 14, 2023
(86) PCT No.: PCT/US2023/083972

§ 371 Date: Jun. 13, 2025

Related U.S. Application Data
(60) Provisional application No. 63/432,700, filed on Dec. 14, 2022.

(51) Int. Cl. C12N 5/09

(52) U.S. Cl. CPC C12N 5/0693; C12N 2513/00; C12N 2533/54

(57) ABSTRACT

Methods, articles, and fluidic systems for sorting and growing organoids are generally provided. Organoids may be useful for various applications, such as certain types of biological studies, or for testing the biocompatibility and performance of drugs. However, conventional methods often struggle to identify, isolate, and use organoids. In some embodiments, using the droplet-based methods and associated articles and fluidic systems provided herein, organoids may be selected, isolated, and studied.

### RELATED APPLICATIONS

This application claims priority under 35 U.S.C. § 119(e) to U.S. Provisional Application No. 63/432,700, filed Dec. 14, 2022, and entitled “ORGANOIDS IN EXTRACELLULAR MATRIX DROPLETS,” which is incorporated herein by reference in its entirety for all purposes.

### TECHNICAL FIELD

Methods of growing and refining organoids, and associated systems and articles are generally described.

### BACKGROUND

An organoid is a miniaturized version of an organ that can be grown in an in vitro environment. Organoids can be grown under certain conditions from one or more cells from a tissue or stem cells. Scientists are interested in organoids for a variety of reasons, for example, because they often exhibit realistic biological structures, and thus may serve as useful biological models. Organoids may be useful for various applications, such as certain types of biological studies, or for testing the biocompatibility and performance of drugs or other substances. However, conventional methods often struggle to identify, isolate, and use organoids. Improvements in organoid techniques are accordingly desired.

### SUMMARY

Methods, articles, and fluidic systems for sorting and growing organoids are generally provided. Organoids may be useful for various applications, such as certain types of biological studies, or for testing the biocompatibility and performance of drugs. However, conventional methods often struggle to identify, isolate, and use organoids. In some embodiments, using the droplet-based methods and associated articles and fluidic systems provided herein, organoids may be selected, isolated, and studied. The subject matter of the present disclosure involves, in some cases, interrelated products, alternative solutions to a particular problem, and/or a plurality of different uses of one or more systems and/or articles.

In one aspect, the present disclosure is generally directed to an article. In one set of embodiments, the article comprises a plurality of cell clusters in a gel, wherein at least 50% of the cell clusters are organoids. In another set of embodiments, the article comprises a plurality of droplets, wherein greater than or equal to 50% of the droplets of the plurality of droplets comprise an organoid.

Another aspect is generally directed to a method. In one set of embodiments, the method comprises growing a plurality of organoids in a first plurality of droplets, and sorting the first plurality of droplets to produce a second plurality of droplets. In some cases, greater than or equal to 50% of the droplets of the second plurality of droplets comprise organoids.

The method, in another set of embodiments, comprises exposing a plurality of droplets containing organoids to an agent suspected of interacting with the organoids, and determining an effect of the agent on the organoids.

In yet another set of embodiments, the method comprises containing an organoid in a droplet containing a gel-forming solution, gelling the gel-forming solution, and growing the organoid to a size exceeding the average diameter of the droplet.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A presents a schematic, cross-sectional illustration of a cell cluster, according to some embodiments;

FIG. 1B presents a schematic, cross-sectional illustration of a non-limiting organoid, according to some embodiments;

FIG. 2A presents a schematic, cross-sectional illustration of a non-limiting method of sorting droplets comprising cell clusters, according to some embodiments;

FIG. 2B presents a schematic, cross-sectional illustration of a non-limiting method of sorting droplets comprising cell clusters, according to some embodiments;

FIG. 3 presents a schematic, cross-sectional illustration of a non-limiting organoid, according to some embodiments;

FIG. 4A presents a schematic, cross-sectional illustration of a non-limiting organoid in a bulk material, according to some embodiments;

FIG. 4B presents a schematic, cross-sectional illustration of a non-limiting organoid in a bulk material, according to some embodiments;

FIG. 5 presents a plurality of droplets comprising organoids, according to some embodiments;

FIG. 6 presents the diameters of droplets within various pluralities of droplets, according to some embodiments;

FIG. 7 presents the growth of exemplary cell clusters within a bulk material, according to some embodiments;

FIGS. 8A-8B present examples of organoids grown inside collagen-matrigel droplets with or without a manual selection at day 5, according to some embodiments;

FIGS. 9A-9D present examples of organoids in droplets that have been sorted, according to some embodiments;

FIGS. 10A-10D present examples of droplets prepared using fluorescent collagen under various conditions, according to some embodiments;

FIGS. 11A-11D present examples of organoids grown inside collagen gel droplets of various sizes and collagen concentrations, according to some embodiments; and

FIGS. 12A-12B present an example of an organoid grown inside a droplet and comprising a luminal structure, according to some embodiments.

### DETAILED DESCRIPTION

Methods, systems, and articles described herein are directed towards the development and refinement of organoids. Organoids may be useful in a laboratory context, where they can, for example, serve as proxies for subjects with a disease or condition, e.g., providing a way to rapidly test the biological effects of an assay or treatment on the disease or condition without the time, cost, or safety constraints associated with animal testing. Unlike the clusters of undifferentiated cells commonly used for similar purposes, organoids include cells that are differentiated to perform different functions within the organoid, in some embodiments, and this may make the organoid a more accurate proxy for a subject than a mere cluster of cells. The present disclosure is directed, in some embodiments, towards improved methods for collecting and growing organoids that may be used for such purposes, or for other purposes.

Initially, a specific, non-limiting embodiment is presented, solely for the purpose of illustration. According to some embodiments, a first plurality of droplets are provided under conditions suitable for the development of organoids. For example, the droplets may include growth factors, gel-formers, and/or other conditions suitable for cell differentiation and organoid growth. In some cases, cells or organoids may be added to the droplets after formation of the droplets; however, in certain embodiments, droplets may be formed around cells or organoids, e.g., that are grown in culture.

At least some of the droplets of the first plurality of droplets may comprise cells. Cells in some droplets of the first plurality of droplets, according to some embodiments, multiply and differentiate to form organoids. Other droplets may merely include cell clusters, suspended cells, or no cells at all. In some embodiments, droplets containing organoids may be separated from droplets that do not contain organoids, e.g., to form a second plurality of droplets. The second plurality of droplets may include, in certain embodiments, a higher concentration of organoids per droplet than the first plurality of droplets. For example, in some embodiments, at least 50% of the droplets of the first plurality of droplets contain organoids. The second plurality of droplets, comprising an elevated concentration of organoids, may then be put to any of a variety of suitable uses. For example, the organoids may be tested within the droplets of the second plurality of droplets. As another example, at least some droplets of the second plurality of droplets may be merged together, or may be added to a gel-forming solution to form a gel that includes a relatively high concentration of organoids, according to some embodiments. Of course, it should be understood that the disclosure is not limited to these specific embodiments, and that any of a variety of techniques may generally be used to produce and study the organoids.

Of course, it should be understood that the forgoing embodiment is provided solely for the purpose of illustration, and is not limiting as other embodiments, described in greater detail below, are also possible.

Organoids may be distinguished from other cell clusters in any of a variety of suitable ways. Like non-organoid cell clusters, organoids can be grown from a single cell, or more than one cell, in some embodiments. However, unlike non-organoid cell clusters (also referred to herein as undifferentiated cell clusters), organoids may include differentiated cells. For example, organoids may include a plurality of cells, wherein some cells of the plurality are adapted to perform a first function, and wherein other cells of the plurality are adapted to perform a second function. Those of ordinary skill in the art will be able to identify whether a population of cells includes differentiated cells. For example, cell differentiation within a plurality of cells may be observed, for example, if a first set of cells of the plurality of cells contain a protein in an amount that significantly differs from the amount of the protein present in a second set of cells of the plurality of cells, or if a first plurality of cells exhibits a first morphology and a second plurality of cells exhibits a second morphology substantially different from the first morphology. In some embodiments, cell differentiation may be observed by its effects. For example, differentiation of cells may give the organoid a morphology that differs from the typical morphology of non-organoid cell clusters, e.g., as described in greater detail below. In addition, in some embodiments, organoids may be determined by determining a product or a function that is not commonly exhibited in undifferentiated cells. Differentiated cells may, of course, differ in a distribution of more than just one protein. In some embodiments, differentiation may be observed based on differences in the concentration of more than one protein within the differentiated cells.

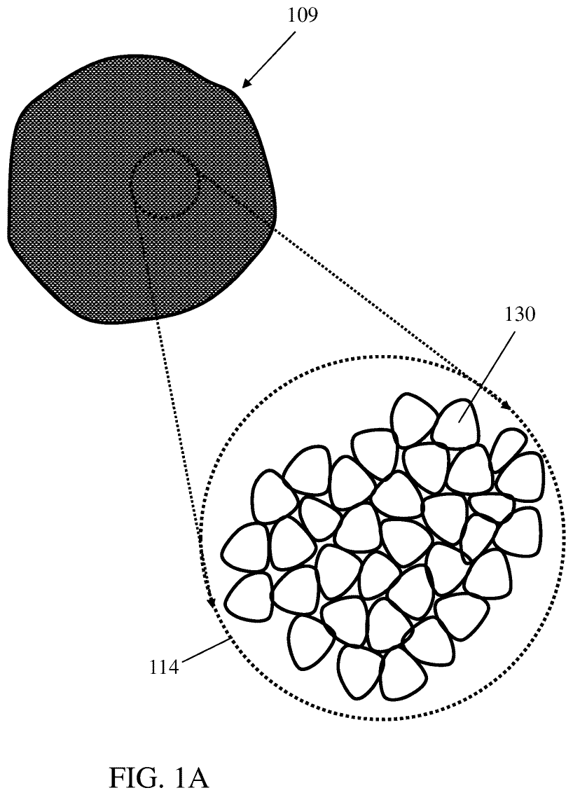

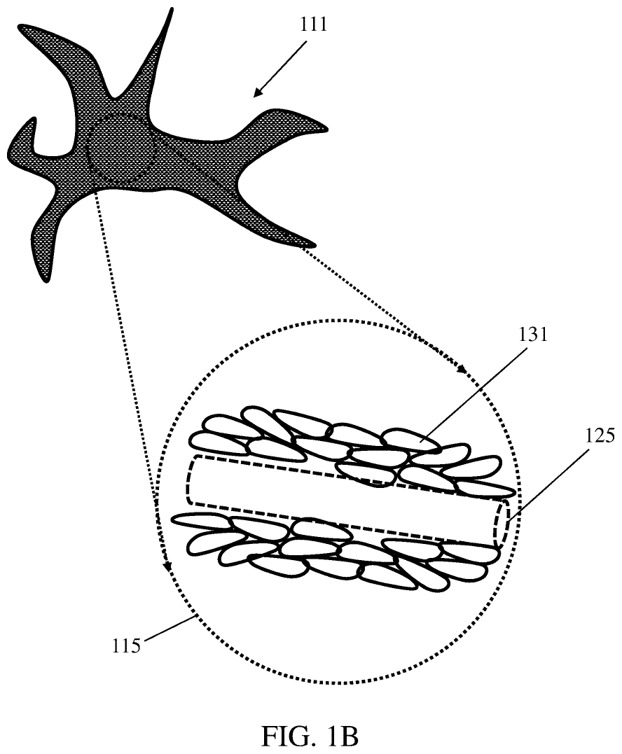

FIG. 1A presents a non-limiting, schematic illustration of a cross-section of undifferentiated cell cluster 109, according to some embodiments. Undifferentiated cell cluster 109 is a spheroid that comprises undifferentiated cells 130 as shown in inset 114, which are densely packed into undifferentiated cluster 109. FIG. 1B, in contrast, presents a non-limiting, schematic illustration of a cross-section of an organoid 111 comprising differentiated cells 131. Organoid 111 is, as a result of the differentiation of its cells, able to support complex morphological features, as illustrated by the branching of organoid 111 and by the presence of tube 125 walled by differentiated cells 131 shown in inset 115, and described in greater detail below. (While tube 125 is shown herein in an idealized configuration, it should be understood that this is for illustrative purposes only, and that the tube may not necessarily be perfectly cylindrical or symmetric.)

More than one organoid may be grown, and indeed, some embodiments are directed towards growing a plurality of organoids. A relatively large portion of the plurality of organoids may be grown from single cells in some cases. For example, in some embodiments, greater than or equal to 50%, greater than or equal to 60%, greater than or equal to 70%, greater than or equal to 80%, greater than or equal to 90%, greater than or equal to 95% of the organoids are grown from single cells. In some embodiments, less than or equal to 100%, less than or equal to 95%, less than or equal to 90%, less than or equal to 80%, less than or equal to 70%, or less than or equal to 60%, of the organoids are grown from single cells. Combinations of these ranges are possible. For example, in some embodiments, greater than or equal to 50% and less than or equal to 100% of the organoids are grown from single cells. Other ranges are also possible.

Any of a variety of appropriate cell types may be used to grow an organoid. For example, in some embodiments an organoid is grown from an animal cell, such as a mammalian cell. The animal cell may, for example, be the cell of a subject such as a human, a rat, a mouse, a dog, a cat, a monkey, or a primate. In some embodiments, the organoid is grown from a healthy cell. However, an organoid is grown using diseased cells, such as cancer cells, in some embodiments. In some embodiments, the cells used to grow the organoids are progenitor cells or stem cells (e.g., induced pluripotent stem cells). Cells such as progenitor cells and stem cells may have a number of advantages for the growth of organoids, including a comparatively high plasticity, which may help them differentiate during organoid development.

Organoids may be particularly advantageous as a cancer model in certain embodiments, since the differentiation of a cancer cell used to grow the organoid may produce a morphology related to the morphology of cancer cells in a tumor of a subject. Thus, organoids grown from cancer cells may be particularly useful as a testing tool for anti-cancer drugs, since the organoids can, more accurately than undifferentiated cells, represent the structure of a tumor and the response of cancer cells in the tumor to exposure to the anti-cancer drug.

Generally, organoids can grow in any of a variety of appropriate media however, it has been recognized in the context of the present disclosure that the growth of organoids within a plurality of droplets may present a number of useful advantages, in certain embodiments. Therefore, in some embodiments, the disclosure is directed towards the growth of a plurality of organoids in a plurality of droplets.

One advantage of growing the organoids in the plurality of droplets is that the droplets can be used to isolate cells used to grow organoids, in some embodiments, limiting the number of organoids that can develop within a single droplet. Furthermore, cells often fail to differentiate as they reproduce, with the result that not every attempt at organoid growth produces an organoid. In some cases, rather, an undifferentiated cluster of cells, instead of an organoid, is produced by the growth process, which may be undesirable in certain cases. Thus, for example, the compartmentalization of the organoids and/or cell clusters within individual droplets of may therefore allow the study of organoids, while unwanted cell clusters may be discarded from the study.

Another advantage of growing organoids within a plurality of droplets is that the droplets can be sorted, in some embodiments. The droplets could be sorted by any of a number of appropriate criteria. For example, in some embodiments, the droplets are sorted based on the presence or absence of an organoid within the droplet. As another example, in some embodiments, droplets are sorted based on the presence or absence of organoids having a particular gene, or expressing a particular protein. In some embodiments, droplets are sorted based on the presence or absence of organoids having a particular morphological feature (as discussed in greater detail below). In some embodiments a first plurality of droplets, wherein some of the droplets comprise organoids, is sorted to produce a second plurality of droplets. The first plurality of droplets of the first plurality of droplets may be sorted to produce any appropriate number of pluralities of droplets (e.g., a second plurality of droplets, a third plurality of droplets, etc.).

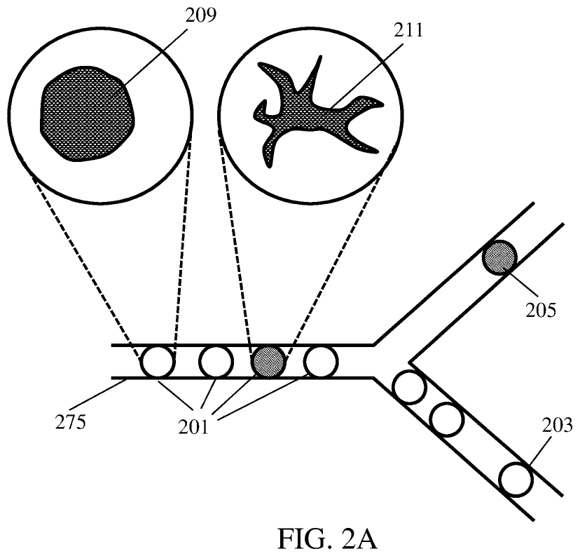

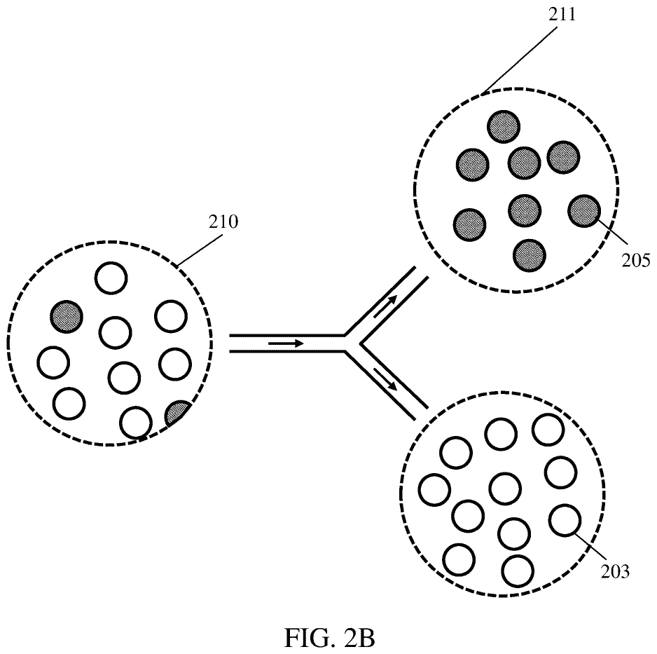

FIGS. 2A-2B provide schematic, side-view illustrations of a non-limiting method of sorting a plurality of droplets, according to some embodiments. As shown in FIG. 2A, a first plurality of droplets 201 may be sorted (e.g., using a microfluidic channel, such as channel 275, or using other microfluidic techniques known to those of ordinary skill in the art). Each droplet 201 in this example can be assessed for the presence of an organoid by any of a variety of appropriate methods. Then the droplets 201 can be individually sorted based on the presence or absence of an organoid. For example, shaded droplets 205 may be likely to include an organoid 211 whereas unshaded droplets 203 may only include undifferentiated cell clusters 209, if they include a cell cluster at all. As shown in FIG. 2B, the end-result of the sorting of the first plurality 210 of droplets may be the formation of a second plurality 225 of droplets 205, a high proportion of which comprise organoids. Droplets 203 that are not sorted into second plurality 210 may be discarded, or may be put to another suitable purpose, as the disclosure is not so limited.

The droplets may be sorted by any of a variety of suitable techniques. For example, in some embodiments the droplets are sorted by measuring an optical property of the droplet, such as a fluorescence, a color change, an absorbance, or a transmission. The optical property may be an innate optical property of the organoid (e.g., a change in absorption or transmission associated with cell differentiation). In some embodiments, the property is produced by the action of an indicator (e.g., a fluorescent or a colorometric indicator) that is added because it experiences a change in optical properties in the presence of an organoid. The sorting may, in some embodiments, be performed by imaging one or more droplets of the plurality of droplets (e.g., using a microscope). For example, the droplets may be imaged to identify a structural characteristic of an organoid (several examples of identifiable structural characteristics of various organoids are described in greater detail below). In some embodiments, the sorting is performed using an acoustic sensor (e.g., to acoustically detect morphological features of the organoid). The sorting may comprise manual sorting of the droplets. In some embodiments, droplets may be sorted by an automatic process, e.g., by determining a threshold value of an optical property or by employing an image processing tool to sort the droplets.

A relatively high proportion of the droplets of the second plurality of droplets may include organoids. In some embodiments, greater than or equal to 50%, greater than or equal to 60%, greater than or equal to 70%, greater than or equal to 80%, greater than or equal to 90%, greater than or equal to 95%, or more of the droplets of the second plurality of droplets include an organoid. In some embodiments, less than or equal to 100%, less than or equal to 95%, less than or equal to 90%, less than or equal to 80%, less than or equal to 70%, less than or equal to 60%, or less of the droplets of the second plurality of droplets include an organoid. Combinations of these ranges are possible. For example, in some embodiments, greater than or equal to 50% and less than or equal to 100% of the droplets of the second plurality of droplets include an organoid. Other ranges are also possible.

In certain embodiments, a droplet of the second plurality of droplets that contains an organoid most commonly contains exactly one organoid. For example, in some embodiments, greater than or equal to 50%, greater than or equal to 60%, greater than or equal to 70%, greater than or equal to 80%, greater than or equal to 90%, greater than or equal to 95%, or more of the droplets of the plurality contain exactly one organoid. In some embodiments, less than or equal to 100%, less than or equal to 95%, less than or equal to 90%, less than or equal to 80%, less than or equal to 70%, less than or equal to 60%, or less of the droplets of the plurality contain exactly one organoid. Combinations of these ranges are possible. For example, in some embodiments, greater than or equal to 50% and less than or equal to 100% of the droplets of the plurality contain exactly one organoid. Other ranges are also possible. In addition, in some cases, populations of droplets containing more than one organoid may also be prepared.

One important feature of organoids is that organoids may develop morphological structures resulting from the differentiation of cells. Without wishing to be bound by any particular theory, an exhaustive list of morphologies associated with organoids is not believed to exist. However, the morphology of organoids typically transcends the tendency of non-organoid cell clusters to form spheroids. By contrast, organoids may include features such as luminal structures, branching, and cavity formation as described in greater detail below.

An organoid comprises a luminal structure, according to some embodiments. Generally, an organoid has a luminal structure if it comprises a tube with walls formed from cells of the organoid. The tube may contain a fluid channel at least partially enclosed by cells of the organoid. As mentioned, the tube may be cylindrical, but more often is only approximately cylindrical. Thus, for example, the tube may be straight, curved, branched, or have other geometries, such as any of those discussed herein. For example, referring back to FIG. 1B, organoid 111 includes a luminal structure represented in inset 115. The luminal structure includes tube 125 walled by cells 131.

Generally, the tube may have any of a variety of appropriate aspect ratios. In some embodiments, an organoid with a luminal structure comprises a tube with an aspect ratio of greater than or equal to 1:3, greater than or equal to 1:5, greater than or equal to 1:10, greater than or equal to 1:20, greater than or equal to 1:50, greater than or equal to 1:100, or greater. In some embodiments, an organoid with a luminal structure comprises a tube with an aspect ratio of less than or equal to 1:1000, less than or equal to 1:500, less than or equal to 1:200, less than or equal to 1:100, less than or equal to 1:50, less than or equal to 1:20, or less. Combinations of these ranges are possible. For example, in some embodiments, an organoid with a luminal structure comprises a tube with an aspect ratio of greater than or equal to 1:3 and less than or equal to 1:1000. Other ranges are also possible.

The tube may have any of a variety of appropriate average diameters. In some embodiments, an organoid with a luminal structure comprises a tube with a diameter of greater than or equal to 5 microns, greater than or equal to 10 microns, greater than or equal to 25 microns, greater than or equal to 50 microns, greater than or equal to 100 microns, greater than or equal to 250 microns, or greater. In some embodiments, an organoid with a luminal structure comprises a tube with a diameter of less than or equal to 500 microns, less than or equal to 250 microns, less than or equal to 100 microns, less than or equal to 50 microns, less than or equal to 25 microns, less than or equal to 10 microns, or less. Combinations of these ranges are possible. For example, in some embodiments, an organoid with a luminal structure comprises a tube with a diameter of greater than or equal to 5 microns and less than or equal to 500 microns. Other ranges are also possible.

The interior of the tube may contain a fluid. The fluid may include a relatively small quantity of cells that are not affixed to a wall of the tube. In some embodiments, cells not affixed to the wall of the tube occupy less than or equal to 20 v/v %, less than or equal to 15 v/v %, less than or equal to 10 v/v %, less than or equal to 8 v/v %, less than or equal to 5 v/v %, less than or equal to 1 v/v %, or less of the tube. Combinations of these ranges are possible. In some embodiments, cells not affixed to the wall of the tube occupy greater than or equal to 0 v/v %, greater than or equal to 1 v/v %, or more of the tube. For example, in some embodiments, cells not affixed to the wall of the tube occupy greater than or equal to 0 v/v % and less than or equal to 20 v/v % of the tube. Other ranges are also possible.

Not every organoid need contain a luminal structure. Generally, in a plurality of organoids, any of a variety of proportions of the plurality of organoids may have luminal structures. For example, in some embodiments, greater than or equal to 0%, greater than or equal to 5%, greater than or equal to 10%, greater than or equal to 25%, greater than or equal to 50%, greater than or equal to 75%, or more organoids of the plurality have luminal structures. In some embodiments, less than or equal to 100%, less than or equal to 75%, less than or equal to 50%, less than or equal to 25%, less than or equal to 10%, or less organoids of the plurality have luminal structures. Combinations of these ranges are possible. For example, in some embodiments, greater than or equal to 0% and less than or equal to 100% organoids of the plurality have luminal structures. Other ranges are also possible.

An organoid may have a relatively low cell number density, relative to an undifferentiated cell cluster. Without wishing to be bound by any particular theory, the relatively low cell number density may be attributed, at least in part, to the growth of structures such as lumina or branches that reduce the sphericity of the organoid. In some embodiments, an organoid can be characterized by two different volumes-its real volume and its reduced volume-and the comparison of these volumes may provide an indication that a cluster of cells is an organoid.

The real volume of an organoid generally refers to the volume situated within an exterior boundary of an organoid or non-differentiated cell cluster, and may be determined, for example, by approximating a boundary of the organoid or non-differentiated cell cluster from a microscopy image and estimating the volume enclosed therein. The reduced volume of an organoid or non-differentiated cell cluster generally refers to the total volume occupied by the organoid or non-differentiated cell cluster's constituent cells, not including the volume of any gaps or spaces between cells. The reduced volume may be determined by estimating the number of cells within an organoid or non-differentiated cell cluster (e.g., based on an optical measurement, such as a fluorescence measurement, wherein the intensity of the measured signal is proportional to the number of cells) and multiplying the number of cells within the organoid by the average volume of the cells of the organoid. For example, the reduced cell volume may be measured by introducing a detection agent (e.g., a fluorophore) into the cells and determining the total volume occupied by the cells using a standard cellular or volumetric concentration of the detection agent.

In some embodiments, cell clusters may be classified by determining a ratio between the real volume of the cell cluster and the reduced volume of the cell cluster. For example, in some embodiments, an organoid has a ratio between its reduced volume and its real volume that is greater than or equal to 2%, greater than or equal to 3%, greater than or equal to 5%, greater than or equal to 10%, greater than or equal to 20%, greater than or equal to 50%, or greater. In some embodiments, an organoid has a ratio between its reduced volume and its real volume that is less than or equal to 80%, less than or equal to 50%, less than or equal to 20%, less than or equal to 10%, less than or equal to 5%, less than or equal to 3%, or less. Combinations of these ranges are possible. For example, in some embodiments, an organoid has a ratio between its reduced volume and its real volume that is greater than or equal to 2% and less than or equal to 80%. Other ranges are also possible.

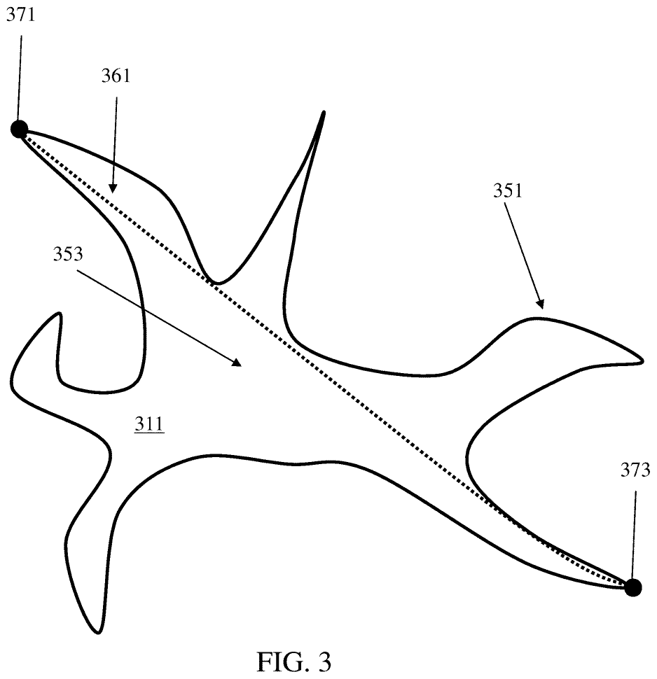

An organoid may have a branched structure, according to some embodiments, wherein the organoid comprises a plurality of arms extending outward from a central nexus. For example, FIG. 3 presents a non-limiting, side-view schematic representation of organoid 311, which is a branched organoid comprising branches 351 and central nexus 353.

In some embodiments, an organoid such as a branched organoid may be characterized in terms of a maximum tip-to-tip path-length. A tip-to-tip pathlength of an organoid or cell cluster generally refers to a shortest path connecting two cells on the surface of an organoid that is entirely contained within the organoid or cell cluster. In a branched organoid, the maximum tip-to-tip path length typically connects tips of two separate branches of the branched organoid. For example, referring again to FIG. 3, organoid 311 has maximum tip-to-tip pathlength 361, which extends from first branch tip 371 to second branch tip 373.

A maximum tip-to-tip pathlength generally refers to the longest tip-to-tip pathlength that can be observed in a given organoid. Generally, an organoid may have any of a variety of maximum tip-to-tip pathlengths. In some embodiments, an organoid has a maximum tip-to-tip pathlength of greater than or equal to 100 microns, greater than or equal to 125 microns, greater than or equal to 150 microns, greater than or equal to 200 microns, greater than or equal to 500 microns, greater than or equal to 1,000 microns, or greater. In some embodiments, an organoid has a maximum tip-to-tip pathlength of less than or equal to 5,000 microns, less than or equal to 2,000 microns, less than or equal to 1,000 microns, less than or equal to 500 microns, less than or equal to 200 microns, less than or equal to 150 microns, or less. Combinations of these ranges are possible. For example, in some embodiments, an organoid has a maximum tip-to-tip pathlength of greater than or equal to 100 microns and less than or equal to 5,000 microns. Other ranges are also possible.

Not every organoid need be branched, and indeed, in some embodiments, no organoid is branched. In some embodiments, within a plurality of organoids, greater than or equal to 0%, greater than or equal to 5%, greater than or equal to 10%, greater than or equal to 25%, greater than or equal to 50%, greater than or equal to 75%, or more of the organoids are branched. In some embodiments, within a plurality of organoids, less than or equal to 100%, less than or equal to 75%, less than or equal to 50%, less than or equal to 25%, less than or equal to 10%, less than or equal to 5%, or less of the organoids are branched. Combinations of these ranges are possible. For example, in some embodiments, within a plurality of organoids, greater than or equal to 0% and less than or equal to 100% of the organoids are branched.

Of course, it should be understood that the aforementioned ranges may be combined (e.g., to say that greater than or equal to 50% of the organoids are branched organoids having a maximum tip-to-tip pathlength of at least 100 microns). Other ranges are also possible, as the disclosure is not so limited.

In some embodiments, organoids may be distinguished from cell clusters based on their non-spheroidal shape. In a perfect sphere, the maximum tip-to-tip pathlength is simply the diameter of the sphere (i.e., (6V/π)^{1/3}, or approximately 1.24 times the cube-root of V, where V is the real volume of the sphere). In contrast, an organoid may have a much longer maximum tip-to-tip pathlength, relative to its real volume (e.g., as a result of having a branched structure). For example, in some embodiments an organoid has a maximum tip-to-tip pathlength of greater than or equal to 1.5×, greater than or equal to 1.8×, greater than or equal to 2×, greater than or equal to 2.2×, greater than or equal to 2.5×, greater than or equal to 3×, or more times the cube-root of the real volume of the organoid. In some embodiments, an organoid has a maximum tip-to-tip pathlength of less than or equal to 5×, less than or equal to 3×, less than or equal to 2.5×, less than or equal to 2.2×, less than or equal to 2×, less than or equal to 1.8×, or less times the cube-root of the real volume of the organoid. Combinations of these ranges are possible. For example, in some embodiments, an organoid has a maximum tip-to-tip pathlength of greater than or equal to 1.5× and less than or equal to 5× times the cube-root of the real volume of the organoid. Other ranges are also possible.

Of course, it should be understood that the aforementioned ranges may be combined (e.g., to say that greater than or equal to 50% of the organoids are branched organoids having a maximum tip-to-tip pathlength of greater than or equal to 1.5× the cube root of a real volume of the organoid). Other ranges are also possible, as the disclosure is not so limited.

In some embodiments, the organoids have other structures. For example, organoids may be budded or spheroidal, depending on the embodiment (although, of course, not every budded, branched, or spheroidal structure is an organoid).

Although manipulation of organoids within droplets is advantageous for any of a number of reasons set forth above, in some embodiments it may be desirable to study the organoid within a bulk material, such as a gel or liquid. For example, growth of an organoid may be limited by the size of a droplet, and placement of the organoid into a bulk material may be desired in order to permit continued growth of the organoid, in some embodiments. According to some embodiments, it may be desirable to situate a plurality of organoids within a common medium (e.g., so that an assay or test can be performed under uniform conditions for each organoid).

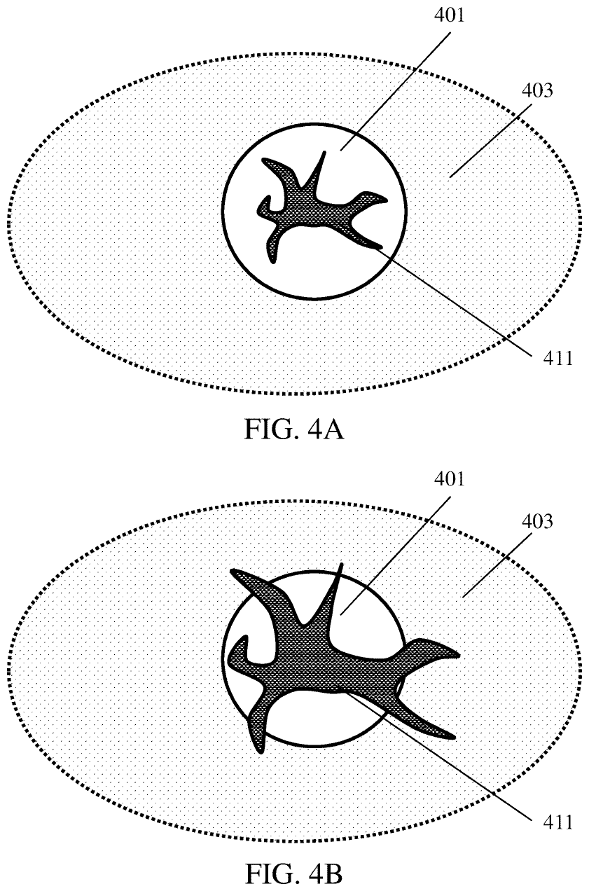

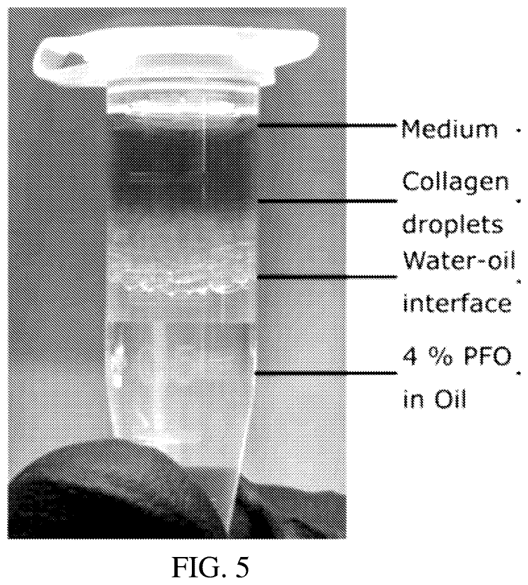

An organoid in a droplet may, in some embodiments, be introduced into and/or grown within a bulk material from a droplet. For example, FIG. 4A presents droplet 401 comprising organoid 411, which has been introduced into bulk material 403. In some embodiments, a droplet such as is discussed herein may contain a gel-forming solution. The gel-forming solution within the droplet may be gelled, in order to form a gel droplet comprising the organoid, according to some embodiments. The gel-forming solution may be gelled under any of a variety of suitable conditions. For example, in some embodiments, the gel-forming solution is gelled at a temperature of greater than or equal to 15° C., greater than or equal to 18° C., greater than or equal to 20° C., greater than or equal to 22° C., greater than or equal to 25° C., greater than or equal to 28° C., greater than or equal to 30° C., greater than or equal to 32° C., greater than or equal to 35° C., greater than or equal to 38° C., greater than or equal to 40° C., or greater than or equal to 42° C. In some embodiments, the gel-forming solution is gelled at a temperature of less than or equal to 45° C., less than or equal to 42° C., less than or equal to 40° C., less than or equal to 38° C., less than or equal to 35° C., less than or equal to 32° C., less than or equal to 30° C., less than or equal to 28° C., less than or equal to 25° C., less than or equal to 22° C., less than or equal to 20° C., or less than or equal to 18° C. Combinations of these ranges are also possible (e.g., greater than or equal to 15° C. and less than or equal to 45° C., greater than or equal to 30° C. and less than or equal to 40° C., or greater than or equal to 35° C. and less than or equal to 38° C.). Other ranges are also possible.

Any of a variety of suitable gel-forming solutions may be used. For example, in some embodiments, the gel-forming solution comprises an extracellular matrix protein (e.g., collagen). In some embodiments, the gel-forming solution comprises a growth-factor. Other elements of the composition of the gel-forming solution, such its salt and pH conditions, may be adjusted by a person of ordinary skill in order to facilitate the maintenance and/or continued growth of the organoid. It should, of course, be understood that an organoid can instead be grown and/or sorted in a droplet that was gelled prior to growth of the organoid, as the disclosure is not so limited.

A gel-forming solution may include an extracellular matrix protein (e.g., collagen) in any of a variety of suitable concentrations. In some embodiments, a gel-forming solution includes an extracellular matrix protein in an amount of greater than or equal to 0.5 mg/mL, greater than or equal to 1 mg/mL, greater than or equal to 1.5 mg/mL, greater than or equal to 2 mg/mL, greater than or equal to 2.5 mg/mL, greater than or equal to 3 mg/mL, greater than or equal to 3.5 mg/mL, greater than or equal to 4 mg/mL, or greater than or equal to 4.5 mg/mL. In some embodiments, a gel-forming solution includes an extracellular matrix protein in an amount of less than or equal to 5 mg/mL, less than or equal to 4.5 mg/mL, less than or equal to 4 mg/mL, less than or equal to 3.5 mg/mL, less than or equal to 3 mg/mL, less than or equal to 2.5 mg/mL, less than or equal to 2 mg/mL, less than or equal to 1.5 mg/mL, or less than or equal to 1 mg/mL. Combinations of these ranges are also possible (e.g., greater than or equal to 0.5 mg/mL and less than or equal to 5 mg/mL, or greater than or equal to 1 mg/mL and less than or equal to 3 mg/mL). Other ranges are also possible.

A gelled droplet may be added to a bulk material in some embodiments, at which point the organoid within the droplet may continue to grow to a size exceeding a diameter of the gelled droplet. For example, FIG. 4B is similar to FIG. 4A, but shows organoid 411 after it has grown to a size exceeding the size of droplet 401.

In some embodiments, the bulk material is also a gel-forming solution that can be gelled. The gel-forming solution of the bulk material may be the same as the gel-forming solution of the droplet, in some embodiments. Alternatively, the gel-forming solution of the bulk material may have a different composition from the gel-forming solution used to gel the droplet, according to some embodiments. The gel-forming solution of the bulk material can then be gelled, in some embodiments, so that the gelled droplet comprising the organoid is encased in gel

As discussed above, one advantage of growing and manipulating the organoids within the droplets is that, in some embodiments, droplets containing organoids can be separated from droplets that merely contain undifferentiated cell clusters, producing an enriched concentration of organoids. In some embodiments, this approach may be used to produce a bulk material comprising a plurality of organoids. A significant advantage of this approach is that it may produce a bulk material that includes an enriched proportion of organoids, relative to the total number of cell clusters in the bulk material. For example, a bulk material such as a gel or gel-forming solution may be produced that it comprises a plurality of cell clusters, wherein greater than or equal to 50%, greater than or equal to 60%, greater than or equal to 70%, greater than or equal to 80%, greater than or equal to 90%, or more of the cell clusters are organoids. In some embodiments, a bulk material such as a gel or gel-forming solution may be produced that it comprises a plurality of cell clusters, wherein less than or equal to 100%, less than or equal to 90%, less than or equal to 80%, less than or equal to 70%, less than or equal to 60%, or less of the cell clusters are organoids. Combinations of these ranges are possible. For example, in some embodiments, a bulk material such as a gel or gel-forming solution may be produced that it comprises a plurality of cell clusters, wherein greater than or equal to 50% and less than or equal to 100% of the cell clusters are organoids. Other ranges are also possible.

An organoid may be used for any of a variety of applications, and may particularly useful for initial testing of treatments or assays. For example, an organoid may, in some embodiments, be exposed to an agent (e.g., a drug or a detection agent) suspected of interacting with the organoid. In some embodiments, an effect of the agent on the organoids may be determined using any of a variety of appropriate methods (e.g., microscopic observation, cytotoxicity testing, agent uptake testing, staining, or any of a variety of other suitable methods of determining the effect of the agent). In some embodiments, the organoid is used for stem cell therapy. Other applications of the organoids are also possible, as the disclosure is not so limited.

As discussed above, in some embodiments the method is directed towards sorting a first plurality of droplets (e.g., based on the presence or absence of an organoid within a droplet). In some embodiments, droplets of the first plurality contains greater than or equal to 5, greater than or equal to 10, greater than or equal to 10², greater than or equal to 10³, greater than or equal to 10⁴, greater than or equal to 10⁵, greater than or equal to 10⁶, greater than or equal to 10⁷, or greater. In some embodiments, droplets of the first plurality contains less than or equal to 10⁸, less than or equal to 10⁸, less than or equal to 10⁷, less than or equal to 10⁶, less than or equal to 10⁵, or less. Combinations of these ranges are possible. For example, in some embodiments, droplets of the first plurality contains greater than or equal to 5 and less than or equal to 10⁸. Other ranges are also possible.

Any suitable method may be chosen to create droplets, and a wide variety of different droplet makers and techniques for forming droplets will be known to those of ordinary skill in the art. For example, a junction of channels may be used to create the droplets. The junction may be, for instance, a T-junction, a Y-junction, a channel-within-a-channel junction (e.g., in a coaxial arrangement, or comprising an inner channel and an outer channel surrounding at least a portion of the inner channel), a cross (or “X”) junction, a flow-focusing junction, or any other suitable junction for creating droplets. See, for example, International Patent Application No. PCT/US2004/010903, filed Apr. 9, 2004, entitled “Formation and Control of Fluidic Species,” by Link, et al., published as WO 2004/091763 on Oct. 28, 2004, or International Patent Application No. PCT/US2003/020542, filed Jun. 30, 2003, entitled “Method and Apparatus for Fluid Dispersion,” by Stone, et al., published as WO 2004/002627 on Jan. 8, 2004, each of which is incorporated herein by reference in its entirety.

Additional details regarding systems and methods for manipulating droplets in a microfluidic system follow, in accordance with certain aspects. For example, various systems and methods for screening and/or sorting droplets are described in U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007, incorporated herein by reference. As a non-limiting example, in some aspects, by applying (or removing) a first electric field (or a portion thereof), a droplet may be directed to a first region or channel; by applying (or removing) a second electric field to the device (or a portion thereof), the droplet may be directed to a second region or channel; by applying a third electric field to the device (or a portion thereof), the droplet may be directed to a third region or channel; etc., where the electric fields may differ in some way, for example, in intensity, direction, frequency, duration, etc.

As mentioned, certain embodiments comprise a droplet contained within a carrying fluid. For example, there may be a first phase forming droplets contained within a second phase, where the surface between the phases comprises one or more proteins. For example, the second phase may comprise oil or a hydrophobic fluid, while the first phase may comprise water or another hydrophilic fluid (or vice versa). It should be understood that a hydrophilic fluid is a fluid that is substantially miscible in water and does not show phase separation with water at equilibrium under ambient conditions (typically 25° C. and 1 atm). Examples of hydrophilic fluids include, but are not limited to, water and other aqueous solutions comprising water, such as cell or biological media, ethanol, salt solutions, saline, blood, etc. In some cases, the fluid is biocompatible.

Similarly, a hydrophobic fluid is one that is substantially immiscible in water and will show phase separation with water at equilibrium under ambient conditions. As previously discussed, the hydrophobic fluid is sometimes referred to by those of ordinary skill in the art as the “oil phase” or simply as an oil. Non-limiting examples of hydrophobic fluids include oils such as hydrocarbons oils, silicon oils, fluorocarbon oils, organic solvents, perfluorinated oils, perfluorocarbons such as perfluoropolyether, etc. Additional examples of potentially suitable hydrocarbons include, but are not limited to, light mineral oil (Sigma), kerosene (Fluka), hexadecane (Sigma), decane (Sigma), undecane (Sigma), dodecane (Sigma), octane (Sigma), cyclohexane (Sigma), hexane (Sigma), or the like. Non-limiting examples of potentially suitable silicone oils include 2 cst polydimethylsiloxane oil (Sigma). Non-limiting examples of fluorocarbon oils include FC3283 (3M), FC40 (3M), Krytox GPL (Dupont), etc. In addition, other hydrophobic entities may be contained within the hydrophobic fluid in some embodiments. Non-limiting examples of other hydrophobic entities include drugs, immunologic adjuvants, or the like.

Thus, the hydrophobic fluid may be present as a separate phase from the hydrophilic fluid. In some embodiments, the hydrophobic fluid may be present as a separate layer, although in other embodiments, the hydrophobic fluid may be present as individual fluidic droplets contained within a continuous hydrophilic fluid, e.g. suspended or dispersed within the hydrophilic fluid. This is often referred to as an oil/water emulsion. The droplets may be relatively monodisperse, or be present in a variety of different sizes, volumes, or average diameters. In some cases, the droplets may have an overall average diameter of less than about 1 mm, or other dimensions as discussed herein. In some cases, a surfactant may be used to stabilize the hydrophobic droplets within the hydrophilic liquid, for example, to prevent spontaneous coalescence of the droplets. Non-limiting examples of surfactants include those discussed in U.S. Pat. Apl. Pub. No. 2010/0105112, incorporated herein by reference. Other non-limiting examples of surfactants include Span80 (Sigma), Span80/Tween-20 (Sigma), Span80/Triton X-100 (Sigma), Abil EM90 (Degussa), Abil we09 (Degussa), polyglycerol polyricinoleate “PGPR90” (Danisco), Tween-85, 749 Fluid (Dow Corning), the ammonium carboxylate salt of Krytox 157 FSL (Dupont), the ammonium carboxylate salt of Krytox 157 FSM (Dupont), or the ammonium carboxylate salt of Krytox 157 FSH (Dupont). In addition, the surfactant may be, for example, a peptide surfactant, bovine serum albumin (BSA), or human serum albumin.

The droplets may have any suitable shape and/or size. In some cases, the droplets may be microfluidic, and/or have an average diameter of less than about 1 mm. For instance, the droplet may have an average diameter of less than about 1 mm, less than about 700 micrometers, less than about 500 micrometers, less than about 300 micrometers, less than about 100 micrometers, less than about 70 micrometers, less than about 50 micrometers, less than about 30 micrometers, less than about 10 micrometers, etc. The average diameter may also be greater than about 5 micrometers, greater than about 7 micrometers, greater than about 10 micrometers, greater than about 30 micrometers, greater than about 50 micrometers, greater than about 70 micrometers, greater than about 100 micrometers, greater than about 300 micrometers, greater than about 500 micrometers, greater than about 700 micrometers, or greater than about 1 mm in some cases. Combinations of any of these are also possible; for example, the diameter of the droplet may be between about 1 mm and about 100 micrometers. The diameter of a droplet, in a non-spherical droplet, may be taken as the diameter of a perfect mathematical sphere having the same volume as the non-spherical droplet.

In some embodiments, the droplets may be of substantially the same shape and/or size (i.e., “monodisperse”), or of different shapes and/or sizes, depending on the particular application. In some cases, the droplets may have a homogenous distribution of cross-sectional diameters, i.e., in some embodiments, the droplets may have a distribution of average diameters such that no more than about 20%, no more than about 10%, or no more than about 5% of the droplets may have an average diameter greater than about 120% or less than about 80%, greater than about 115% or less than about 85%, greater than about 110% or less than about 90%, greater than about 105% or less than about 95%, greater than about 103% or less than about 97%, or greater than about 101% or less than about 99% of the average diameter of the microfluidic droplets. Some techniques for producing homogenous distributions of cross-sectional diameters of droplets are disclosed in International Patent Application No. PCT/US2004/010903, filed Apr. 9, 2004, entitled “Formation and Control of Fluidic Species,” by Link, et al., published as WO 2004/091763 on Oct. 28, 2004, incorporated herein by reference. In addition, in some instances, the coefficient of variation of the average diameter of the droplets may be less than or equal to about 20%, less than or equal to about 15%, less than or equal to about 10%, less than or equal to about 5%, less than or equal to about 3%, or less than or equal to about 1%. However, in other embodiments, the droplets may not necessarily be substantially monodisperse, and may instead exhibit a range of different diameters.

Those of ordinary skill in the art will be able to determine the average diameter of a population of droplets, for example, using laser light scattering or other known techniques. The droplets so formed can be spherical, or non-spherical in certain cases. The diameter of a droplet, in a non-spherical droplet, may be taken as the diameter of a perfect mathematical sphere having the same volume as the non-spherical droplet.

In some embodiments, one or more droplets may be created within a channel by creating an electric charge on a fluid surrounded by a liquid, which may cause the fluid to separate into individual droplets within the liquid. In some embodiments, an electric field may be applied to the fluid to cause droplet formation to occur. The fluid can be present as a series of individual charged and/or electrically inducible droplets within the liquid. Electric charge may be created in the fluid within the liquid using any suitable technique, for example, by placing the fluid within an electric field (which may be AC, DC, etc.), and/or causing a reaction to occur that causes the fluid to have an electric charge.

The electric field, in some embodiments, is generated from an electric field generator, i.e., a device or system able to create an electric field that can be applied to the fluid. The electric field generator may produce an AC field (i.e., one that varies periodically with respect to time, for example, sinusoidally, sawtooth, square, etc.), a DC field (i.e., one that is constant with respect to time), a pulsed field, etc. Techniques for producing a suitable electric field (which may be AC, DC, etc.) are known to those of ordinary skill in the art. For example, in one embodiment, an electric field is produced by applying voltage across a pair of electrodes, which may be positioned proximate a channel such that at least a portion of the electric field interacts with the channel. The electrodes can be fashioned from any suitable electrode material or materials known to those of ordinary skill in the art, including, but not limited to, silver, gold, copper, carbon, platinum, copper, tungsten, tin, cadmium, nickel, indium tin oxide (“ITO”), etc., as well as combinations thereof.

In another set of embodiments, droplets of fluid can be created from a fluid surrounded by a liquid within a channel by altering the channel dimensions in a manner that is able to induce the fluid to form individual droplets. The channel may, for example, be a channel that expands relative to the direction of flow, e.g., such that the fluid does not adhere to the channel walls and forms individual droplets instead, or a channel that narrows relative to the direction of flow, e.g., such that the fluid is forced to coalesce into individual droplets. In some cases, the channel dimensions may be altered with respect to time (for example, mechanically or electromechanically, pneumatically, etc.) in such a manner as to cause the formation of individual droplets to occur. For example, the channel may be mechanically contracted (“squeezed”) to cause droplet formation, or a fluid stream may be mechanically disrupted to cause droplet formation, for example, through the use of moving baffles, rotating blades, or the like.

A droplet comprising a gel-forming solution may be formed or manipulated at any of a variety of appropriate temperatures. In particular, it may be advantageous, according to some embodiments, to form or manipulate droplets at a relatively low temperature. For example, in some embodiments, a droplet comprising a gel-forming solution is formed and/or manipulated at a temperature of less than or equal to 25° C., less than or equal to 22° C., less than or equal to 20° C., less than or equal to 18° C., less than or equal to 15° C., less than or equal to 12° C., less than or equal to 10° C., less than or equal to 8° C., less than or equal to 5° C., or less than or equal to 2° C. In some embodiments, a droplet comprising a gel-forming solution is formed and/or manipulated at a temperature of greater than or equal to 1 C, greater than or equal to 2° C., greater than or equal to 5° C., greater than or equal to 8° C., greater than or equal to 10° C., greater than or equal to 12° C., greater than or equal to 15° C., greater than or equal to 18° C., greater than or equal to 20° C., or greater than or equal to 22° C. Combinations of these ranges are also possible (e.g., greater than or equal to 1° C. and less than or equal to 25° C., greater than or equal to 1° C. and less than or equal to 10° C., or greater than or equal to 2° C. and less than or equal to 5° C.). Other ranges are also possible.

Some embodiments generally relate to systems and methods for fusing or coalescing two or more droplets into one droplet, e.g., where the two or more droplets ordinarily are unable to fuse or coalesce, for example, due to composition, surface tension, droplet size, the presence or absence of surfactants, etc. In certain cases, the surface tension of the droplets, relative to the size of the droplets, may also prevent fusion or coalescence of the droplets from occurring.

As a non-limiting example, two droplets can be given opposite electric charges (i.e., positive and negative charges, not necessarily of the same magnitude), which can increase the electrical interaction of the two droplets such that fusion or coalescence of the droplets can occur due to their opposite electric charges. For instance, an electric field may be applied to the droplets, the droplets may be passed through a capacitor, a chemical reaction may cause the droplets to become charged, etc. The droplets, in some cases, may not be able to fuse even if a surfactant is applied to lower the surface tension of the droplets. However, if the droplets are electrically charged with opposite charges (which can be, but are not necessarily of, the same magnitude), the droplets may be able to fuse or coalesce. As another example, the droplets may not necessarily be given opposite electric charges (and, in some cases, may not be given any electric charge), and are fused through the use of dipoles induced in the droplets that causes the droplets to coalesce. Also, the two or more droplets allowed to coalesce are not necessarily required to meet “head-on.” Any angle of contact, so long as at least some fusion of the droplets initially occurs, is sufficient. See also, e.g., U.S. patent application Ser. No. 11/698,298, filed Jan. 24, 2007, entitled “Fluidic Droplet Coalescence,” by Ahn, et al., published as U.S. Patent Application Publication No. 2007/0195127 on Aug. 23, 2007, incorporated herein by reference in its entirety.

In one set of embodiments, a fluid may be injected into a droplet. The fluid may be microinjected into the droplet in some cases, e.g., using a microneedle or other such device. In other cases, the fluid may be injected directly into a droplet using a fluidic channel as the droplet comes into contact with the fluidic channel. Other techniques of fluid injection are disclosed in, e.g., International Patent Application No. PCT/US2010/040006, filed Jun. 25, 2010, entitled “Fluid Injection,” by Weitz, et al., published as WO 2010/151776 on Dec. 29, 2010; or International Patent Application No. PCT/US2009/006649, filed Dec. 18, 2009, entitled “Particle-Assisted Nucleic Acid Sequencing,” by Weitz, et al., published as WO 2010/080134 on Jul. 15, 2010, each incorporated herein by reference in its entirety.

The following documents are each incorporated herein by reference in its entirety for all purposes: Int. Pat. Apl. Pub. No. WO 2016/168584, entitled “Barcoding System for Gene Sequencing and Other Applications,” by Weitz et al.; Int. Pat. Apl. Pub. No. WO 2015/161223, entitled “Methods and Systems for Droplet Tagging and Amplification,” by Weitz, et al.; U.S. Pat. Apl. Ser. No. 61/980,541, entitled “Methods and Systems for Droplet Tagging and Amplification,” by Weitz, et al.; U.S. Pat. Apl. Ser. No. 61/981,123, entitled “Systems and Methods for Droplet Tagging,” by Bernstein, et al.; Int. Pat. Apl. Pub. No. WO 2004/091763, entitled “Formation and Control of Fluidic Species,” by Link et al.; Int. Pat. Apl. Pub. No. WO 2004/002627, entitled “Method and Apparatus for Fluid Dispersion,” by Stone et al.; Int. Pat. Apl. Pub. No. WO 2006/096571, entitled “Method and Apparatus for Forming Multiple Emulsions,” by Weitz et al.; Int. Pat. Apl. Pub. No. WO 2005/021151, entitled “Electronic Control of Fluidic Species,” by Link et al.; Int. Pat. Apl. Pub. No. WO 2011/056546, entitled “Droplet Creation Techniques,” by Weitz, et al.; Int. Pat. Apl. Pub. No. WO 2010/033200, entitled “Creation of Libraries of Droplets and Related Species,” by Weitz, et al.; U.S. Pat. Apl. Pub. No. 2012-0132288, entitled “Fluid Injection,” by Weitz, et al.; Int. Pat. Apl. Pub. No. WO 2008/109176, entitled “Assay And Other Reactions Involving Droplets,” by Agresti, et al.; and Int. Pat. Apl. Pub. No. WO 2010/151776, entitled “Fluid Injection,” by Weitz, et al.; and U.S. Pat. Apl. Ser. No. 62/072,944, entitled “Systems and Methods for Barcoding Nucleic Acids,” by Weitz, et al.

In addition, the following are incorporated herein by reference in their entireties: U.S. Pat. Apl. Ser. No. 61/981,123 filed Apr. 17, 2014; PCT Pat. Apl. Ser. No. PCT/US2015/026338, filed Apr. 17, 2015, entitled “Systems and Methods for Droplet Tagging”; U.S. Pat. Apl. Ser. No. 61/981,108 filed Apr. 17, 2014; U.S. Pat. Apl. Ser. No. 62/072,944, filed Oct. 30, 2014; PCT Pat. Apl. Ser. No. PCT/US2015/026443, filed on Apr. 17, 2015, entitled “Systems and Methods for Barcoding Nucleic Acids”; U.S. Pat. Apl. Ser. No. 62/106,981, entitled “Systems, Methods, and Kits for Amplifying or Cloning Within Droplets,” by Weitz, et al.; U.S. Pat. Apl. Pub. No. 2010-0136544, entitled “Assay and Other Reactions Involving Droplets,” by Agresti, et al.; U.S. Pat. Apl. Ser. No. 61/981,108, entitled “Methods and Systems for Droplet Tagging and Amplification,” by Weitz, et al.; Int. Pat. Apl. Pub. No. PCT/US2014/037962, filed May 14, 2014, entitled “Rapid Production of Droplets,” by Weitz, et al.; and U.S. Provisional Patent Application Ser. No. 62/133,140, filed Mar. 13, 2015, entitled “Determination of Cells Using Amplification,” by Weitz, et al.

In some embodiments, the droplets are broken down after amplification, e.g., to allow the amplified nucleic acids to be pooled together, or to remove the droplets from an oil. A wide variety of methods for “breaking” or “bursting” droplets are available to those of ordinary skill in the art. For example, droplets contained in a carrying fluid may be disrupted using techniques such as mechanical disruption, chemical disruption, or ultrasound. Droplets may also be disrupted using chemical agents or surfactants, for example, 1H,1H,2H,2H-perfluorooctanol.

In some cases, one or more of the droplets may be uniquely identified or “barcoded,” e.g., such that a droplet can be distinguished from other droplets. Examples of identification technologies include, but are not limited to, unique nucleic acid strands, fluorescent particles, or the like. In some cases, the barcodes can be sequenced, e.g., to determine or identify a droplet, an organoid contained within a droplet, or the like. Examples of barcoding approaches include, but are not limited to, US Pat. Apl. Pub. Nos. 2013/0274117, 2015/0298091, 2017/0029813, or 2018/0087078.

In some embodiments, a plurality of identification elements may be chosen to identify droplets such that there are at least 3 distinguishable identification elements, at least 4 distinguishable identification elements, at least 6 distinguishable identification elements, at least 8 distinguishable identification elements, at least 9 distinguishable identification elements, at least about 10 distinguishable identification elements, at least about 20 distinguishable identification elements, at least about 30 distinguishable identification elements, at least about 40 distinguishable identification elements, at least about 50 distinguishable identification elements, at least about 60 distinguishable identification elements, at least about 70 distinguishable identification elements, at least about 80 distinguishable identification elements, at least about 90 distinguishable identification elements, at least about 100 distinguishable identification elements, etc. in a droplet.

One non-limiting example of a plurality of distinguishable identification elements are the Luminex® FlexMAP Microspheres beads commercially available from Luminex® Corp. Beads or particles such as these may be distinguished, according to one embodiment, by the use of two or more dyes or other compounds that can be independently varied within each bead or particle. A plurality of distinguishable beads may be used as a plurality of identification elements, according to certain embodiments. As another, specific non-limiting example, particles comprising polystyrene and one or more dyes may be used as identification elements. The dyes employed within the particles may include, for instance, squaric acid-based molecules or other fluorescent molecules that exhibit fluorescence, e.g., extending into near infrared and/or infrared region. In some cases, two or more dyes with concentrations that can be independently controlled can be used within each particle.

The following examples are intended to illustrate certain embodiments of the present disclosure, but do not exemplify the full scope of the disclosure.

### EXAMPLE 1

This example describes the growth of organoids in a gel-forming solution. Organoids were grown in a gel-forming solution produced by preparing a cell media comprising 1.3 mg/mL collagen (an extracellular matrix protein) by adding 10× phosphate buffered saline (PBS) and 1×4-(2-hydroxyethyl)-1-piperazineethanesulfonic acid (HEPES) buffer to the collagen in separate steps. The total buffer volume was one tenth of the volume of the collagen concentrate, so that the buffer formed 1/11^{th} of the final cell media. Next, a cell solution was added to the cell media and the solutions were thoroughly mixed. The pH of the resulting solution was around 7. To avoid premature gelling, the resulting solution was kept at 4° C. during preparation. Next, a microfluidic system was used to form the solution into a plurality of droplets having diameters of 300-500 microns that were then incubated at 37° C. for 1 h to gel the gel-forming solution.

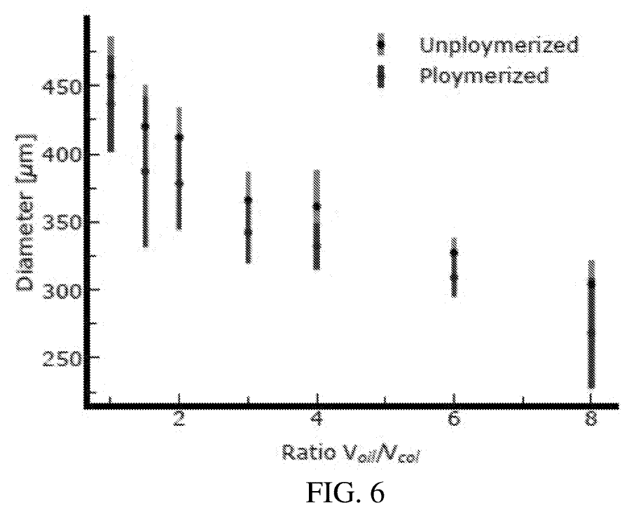

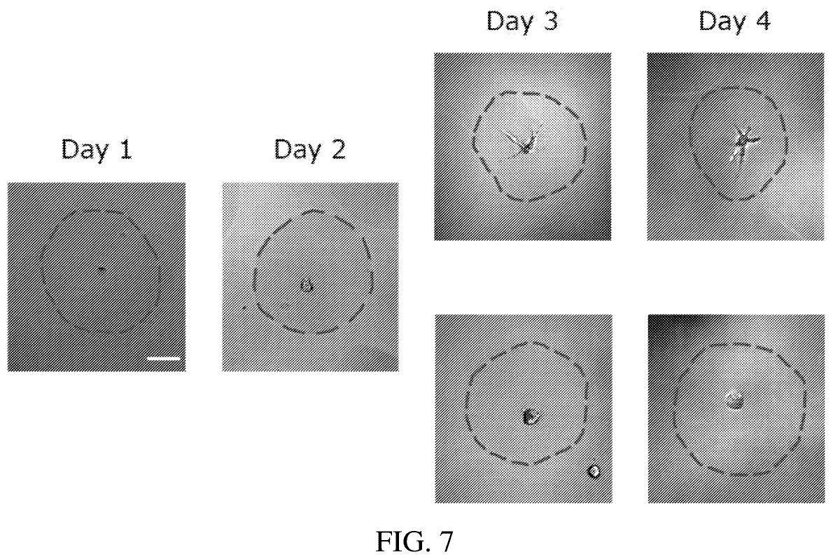

Next, the plurality of droplets was broken to recover the droplets. The droplets were then washed 3 times with 1,1,1,2,3,4,4,5,5,6,6,6-Dodecafluoro-2-(trifluoromethyl)hexan-3-yl ethyl ether (HFE 7500) to wash away the surfactant, and the HFE7500 was replaced with HFE 7500/4% 1H,1H,2H,2H-perfluorooctanol (PFO) and Dulbecco's Modified Eagle's Medium (DMEM), with 0.1 w/w % Tween 80 added on top of the oil to emulsify the plurality of droplets, improving their ability to transfer to the aqueous phase, and resulting in separation of the plurality of droplets. FIG. 5 presents a photograph of a non-limiting plurality of gelled droplets formed by this method, which have been separated from oil. FIG. 6 presents the diameter of the droplets versus the volume ratio of the oil-to-collogen solution, demonstrating the precise control of diameters of the droplets of the plurality.

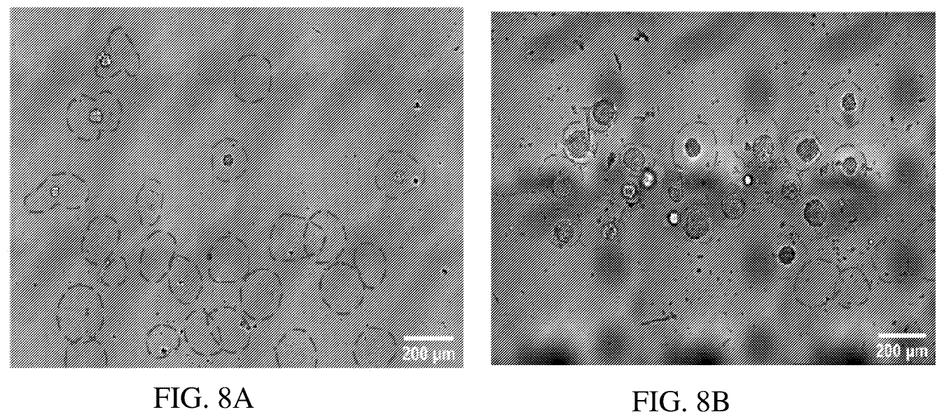

After 5 minutes, the separated droplets were transferred into a second gel-forming solution and permitted to grow. FIG. 7 presents microscope images of the growth of the organoids within the second gel-forming solution. Cell clusters initially appeared relatively spherical, but after two days, some of the spherical clusters differentiated to form organoids (top-row) that continued to grow, while some continued to grow as undifferentiated spheroidal clusters (bottom row). After 4 days, the organoids grown by this method grew to a size exceeding the diameter of their initial droplets. Although no method of sorting was used in this example, this example demonstrates that droplets comprising organoids may be added to a gel-forming solution to allow the organoids to grow to a size exceeding the size of the droplets.

### EXAMPLE 2

This prophetic example demonstrates the sorting of droplets based on the presence or absence of an organoid within the droplet. In this example, a plurality of droplets comprising cell clusters is formed and sorted using a combination of sensors configured to detect differences in cluster morphology based on changes in fluorescence, differences in acoustic signal, and differences in images collected from the droplets. The system has been trained to recognizes differences between organoids and other cell clusters, such as presence of branches, number of branches, or cell density, and to sort droplets including organoids with the desired characteristics into a second plurality of droplets, which includes a high concentration of the desired organoids. The second plurality of organoids are then used to perform drug testing.

### EXAMPLE 3

In this example, pancreatic ductal adenocarcinoma cells were incorporated into collagen-matrigel droplets. The collagen-matrigel mix was prepared such that 80% 1.3 mg/ml collagen was mixed with 20% growth factor reduced matrigel (R&D Systems). The cells were allowed to proliferate for 5 days in the droplets. When cultured in a collagen-matrigel mix PDAC cells display reduced invasion into the surrounding tissue and tend to rather grow in a spherical structure. Therefore, the presence of a spheroid in a single droplet was used for sorting. Cells that did not proliferate inside the droplets, spheroids that grew into neighboring droplets, as well as empty droplets were considered to not meet this selection criterion. For imaging, the cells were stained with a Hoechst live cell stain to visualize the cell nucleus.

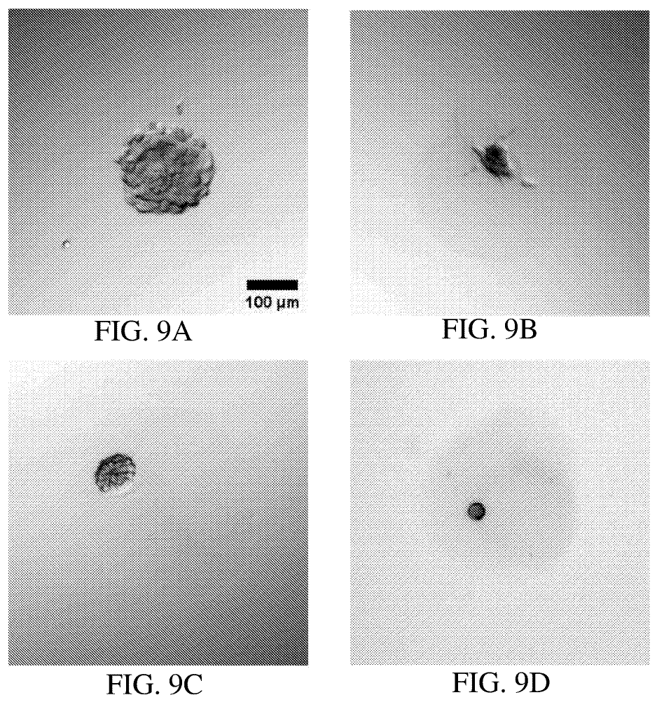

FIG. 8A shows droplets that have not been selected. The displayed droplets have a high fraction of droplets that did not meet the selection criterion. Those droplets are surrounded by a contour. The droplets with a spheroid are highlighted by a lighter contour. To increase the fraction of droplets that correspond to the selection criterion droplets were manually selected by aspirating them with a pipette. FIG. 8B shows droplets that had been manually selected. The fraction of droplets meeting the selection criterion was higher compared to the unselected droplets from FIG. 8A. Thus, by screening the droplets for droplets that meet certain parameters, it was possible to obtain a subset of droplets with a higher fraction of droplets with that parameter.

FIG. 8 shows organoids grown inside collagen-matrigel droplets with or without manual selection at day 5. The cells have been stained with a Hoechst nuclear stain. FIG. 8A shows organoids in collagen-matrigel droplets that have not been sorted. Without sorting many droplets are empty, contain dead cells or a spheroid that grew into a neighboring droplet. FIG. 8B shows organoids in collagen-matrigel droplets that have been manually selected. Compared to the previous figure, the fraction of droplets containing a correctly grown spheroid was higher.

### EXAMPLE 4

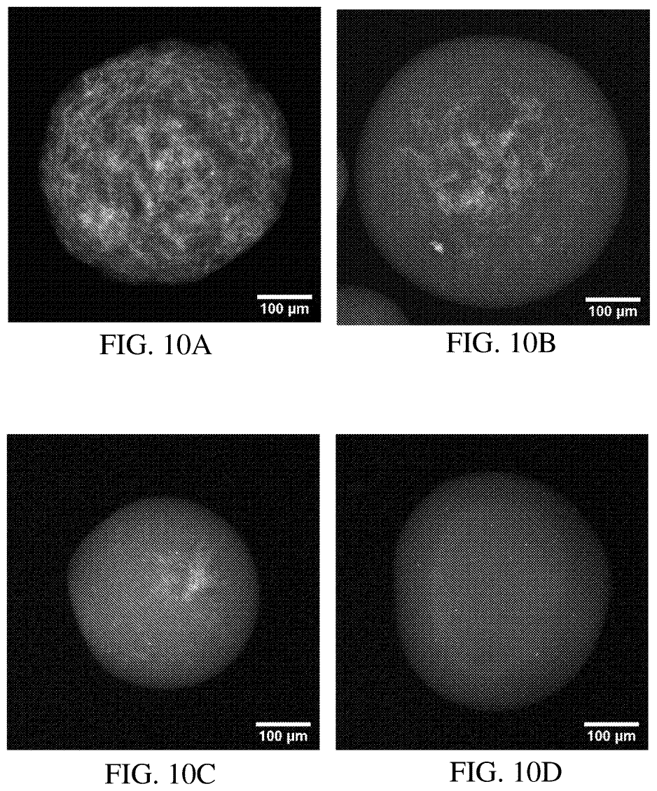

In this example, pancreatic ductal adenocarcinoma cells were cultured in collagen droplets for 4 days. At day 4 the cells were stained using a GFP live cell stain to fluorescently label the cytoplasm. After staining the cells, the droplets where washed with PBS with 10% FBS (fetal bovine serum) to remove the staining solution. The cells were diluted to 500 droplets per milliliter and sorted using the Biosorter from Union Biometrica with a 1000 micrometer diameter nozzle. The sorted droplets were collected in a 96 well plate and imaged after sorting. FIG. 9 displays a selection of the sorted droplets. Thus, sorting based on the fluorescent signal of the spheroids inside the collagen droplets was demonstrated.

### EXAMPLE 5

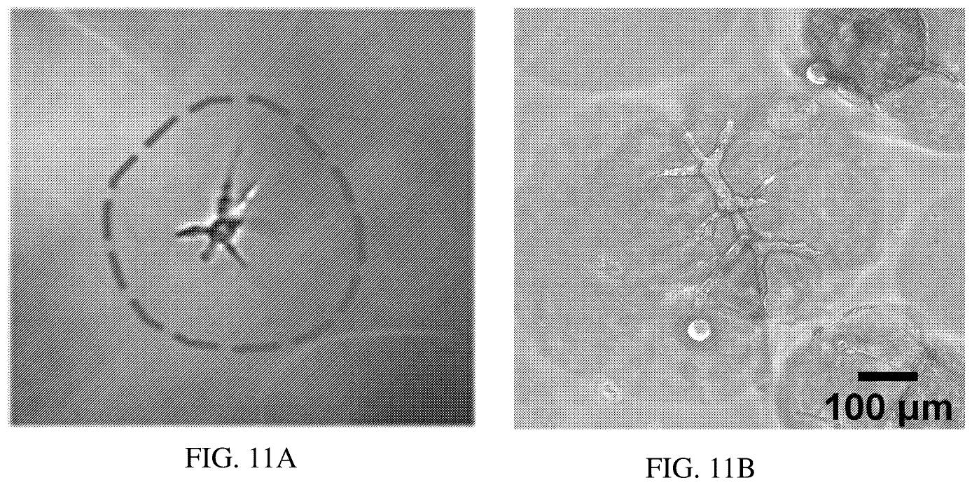

In this example, the importance of the temperature for droplet handling of gel forming droplets comprising extracellular matrix proteins (in particular, collagen) was studied by varying the temperature of (i) droplet formation and handling and (ii) gel formation. Droplets comprising fluorescently labelled the collagen were prepared so that collagen density variation created contrast. Droplets prepared under 4 different conditions were tested: (a) droplet handling at 21° C. and gel formation at 21° C.; (b) droplet handling at 21° C. and gel formation at 4° C.; (c) droplet handling at 21° C. and gel formation at 4° C.; and (d) droplet handling at 4° C. and gel formation at 37° C. Gelled droplets formed by each method are shown in the micrographs presented in FIGS. 10A-10D, respectively. High density variation, corresponding to collagen fiber formation, was observed in samples (a)-(c) but was not observed in sample (d), as shown in FIG. 10D. These results demonstrate that keeping the droplets cool until gel formation is intended, followed by heating of the droplets to relatively high temperatures (e.g., 37° C.) produced the smoothest gels believed to be best suited for organoid growth. Under the other conditions, the collagen was observed to polymerize to form aggregated fibers, represented as the bright patches in FIGS. 10A-10C.

### EXAMPLE 6

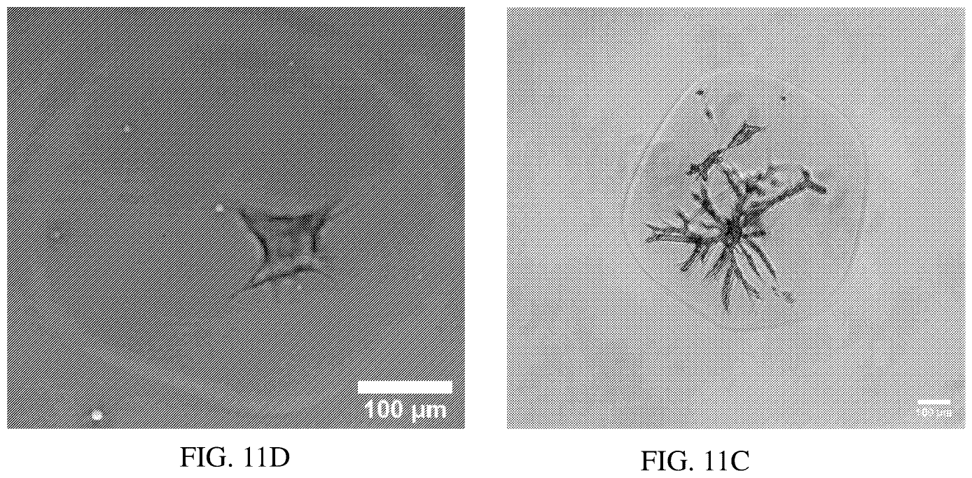

This example demonstrates morphological variation in organoids formed in droplets of various sizes and collagen compositions. Four different combinations of droplet sizes and collagen concentrations: (a) <500 micrometer droplets comprising 1.3 mg/mL collagen; (b) >500 micrometer droplets comprising 1.3 mg/mL collagen; (c) <500 micrometer droplets comprising 3 mg/mL collagen; and (d) >500 micrometer droplets comprising 3 mg/mL collagen. FIGS. 11A-11D present examples of branched organoids observed in each droplet type. Although branched structures grew in all 4 tested conditions, qualitatively, bigger droplets and a higher collagen concentration were observed to generally be associated with more branched organoids and larger organoids, relative to smaller droplets or lower collagen concentrations.

### EXAMPLE 7

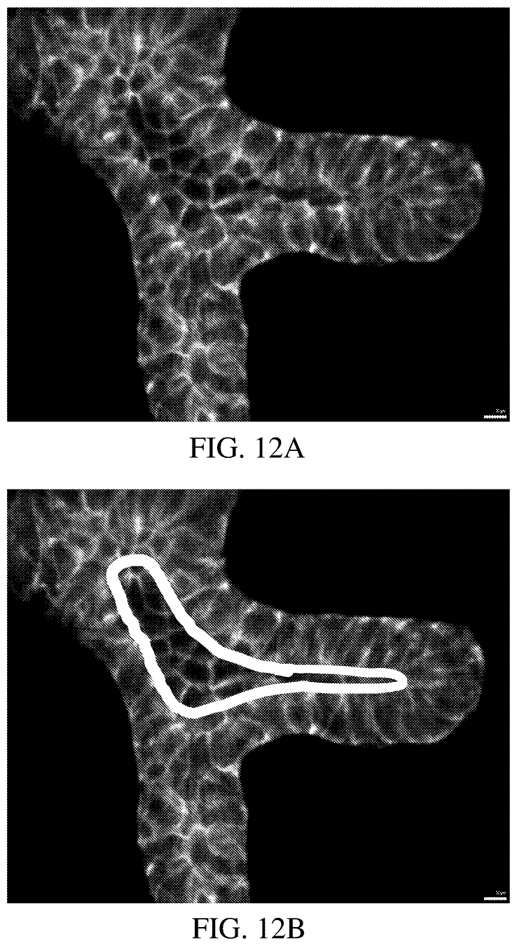

This example illustrates the formation of a luminal structure in an organoid formed within a droplet. Organoids were prepared in droplets containing gelled collagen (3 mg/mL) and using PDAC cells expressing E-Cadherin labeled with green fluorescent protein (GFP) and LifeAct labeled with mCherry. FIG. 12A presents a micrograph illustrating fluorescence of the two labels in an exemplary organoid. A hollow structure is clearly visible within the droplet. FIG. 12B presents a reproduction of the micrograph of FIG. 12A, whereon a curved, white line has been superimposed to illustrate the surface of the luminal structure.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
growing a plurality of organoids in a first plurality of droplets; and
sorting the first plurality of droplets to produce a second plurality of droplets wherein greater than or equal to 50% of the droplets of the second plurality of droplets comprise organoids.

2. A method, comprising:
exposing a plurality of droplets containing organoids to an agent suspected of interacting with the organoids; and
determining an effect of the agent on the organoids.

3. The method of any one of the preceding claims, wherein at least 50% of the organoids have a luminal structure.

4. The method of any one of the preceding claims, wherein at least 50% of the organoids have a ratio of real volume to reduced volume that is greater than or equal to 125%.

5. The method of any one of the preceding claims, wherein greater than or equal to 50% of the organoids are branched organoids having a maximum tip-to-tip pathlength of at least 100 microns.

6. The method of any one of the preceding claims, wherein at least 50% of the organoids have a maximum tip-to-tip pathlength that is greater than or equal to 1.5× a cube-root of a real volume of the organoid.

7. The method of any one of the preceding claims, wherein greater than or equal to 50% of the organoids are branched organoids.

8. The method of any one of the preceding claims, wherein the maximum tip to tip path length is greater than or equal to 125 microns.

9. The method of any one of the preceding claims, wherein the organoid comprises cancer cells.

10. The method of any one of the preceding claims, wherein the organoid comprises human cells.

11. The method of any one of the preceding claims, wherein the method comprises growing greater than or equal to 50% of the organoids from single cells.

12. The method of any one of the preceding claims, wherein the sorting is performed at least in part by imaging at least some droplets of the plurality of droplets.

13. The method of any one of the preceding claims, wherein the sorting is performed at least in part by measuring an optical property of at least some droplets of the plurality of droplets.

14. The method of any one of the preceding claims, wherein the sorting of the organoids is at least partially based on a presence or absence of a particular gene or protein of the organoid, the presence or absence of a particular morphological feature of the organoid, and/or a distribution of a protein within the organoid.

15. The method of any one of the preceding claims, further comprising sorting the first plurality of droplets to produce a third plurality of droplets wherein greater than or equal to 50% of the droplets of the third plurality of droplets comprise organoids, wherein at least 50% of the organoids of the third plurality of droplets differ from at least 50% of the organoids of the second plurality of droplets based on a difference in the presence or absence of a particular gene or protein or the presence or absence of a particular morphological feature.

16. A method, comprising:
containing an organoid in a droplet containing a gel-forming solution;
gelling the gel-forming solution; and
growing the organoid to a size exceeding the average diameter of the droplet.

17. The method of claim 16, wherein the gel-forming solution comprises an extracellular matrix protein.

18. The method of any one of claims 16 or 17, wherein the gel-forming matrix comprises collagen.

19. The method of any one of claims 16-18, wherein the gel-forming matrix comprises one or more growth factors.

20. The method of any one of claims 16-19, wherein the gel-forming material is a first gel-forming material, and wherein the step of growing the organoid to the size exceeding the diameter of the droplet is performed by immersing the gelled droplet in a second gel-forming material.

21. An article, comprising:
a plurality of cell clusters in a gel, wherein at least 50% of the cell clusters are organoids.

22. An article comprising:
a plurality of droplets, wherein greater than or equal to 50% of the droplets of the plurality of droplets comprise an organoid.

23. The article of any one of claims 21 or 22, wherein at least 50% of the organoids have a luminal structure.

24. The article of any one of claims 21-23, wherein at least 50% of the organoids have a ratio of real volume to reduced volume that is greater than or equal to 125%.

25. The article of any one of claims 21-24, wherein greater than or equal to 50% of the organoids are branched organoids having a maximum tip-to-tip pathlength of at least 100 microns.

26. The article of any one of claims 21-25, wherein at least 50% of the organoids have a maximum tip-to-tip pathlength that is greater than or equal to 1.5× a cube-root of a real volume of the organoid.

27. The article of any one of claims 21-26, wherein greater than or equal to 50% of the organoids are branched organoids.

28. The article of any one of claims 21-27, wherein the maximum tip to tip path length is greater than or equal to 125 microns.

29. The article of any one of claims 21-28, wherein the organoid comprises cancer cells.

30. The article of any one of claims 21-29, wherein the organoid comprises human cells.
